(19) United States
(12) Patent Application Publication
(10) Pub. No.: US 2026/0283913 A1
(43) Pub. Date: Sep. 24, 2026

(54) METAL AQUO-COMPLEX MEDIATED SYNTHESIS OF ZINC OXIDE-BASED STRUCTURES

(71) Applicant: Zinco Verde Inc., Cambridge, MA (US)

(72) Inventors: Jacopo Samson, Philadelphia, PA (US); John W. Harrold, Cambridge, MA (US)

(21) Appl. No.: 19/473,948

(22) PCT Filed: Apr. 9, 2024
(86) PCT No.: PCT/US24/23675

§ 371 Date: Oct. 9, 2025

Related U.S. Application Data
(60) Provisional application No. 63/495,281, filed on Apr. 10, 2023.

(51) Int. Cl. A61K 8/02; A61K 8/27; A61Q 19/00; C01G 9/02

(52) U.S. Cl. CPC A61K 8/0241; A61K 8/27; C01G 9/02; A61K 2800/651; A61Q 19/00; C01P 2002/52; C01P 2002/54; C01P 2004/03; C01P 2004/04; C01P 2004/32; C01P 2004/51; C01P 2004/61; C01P 2004/62; C01P 2004/64

(57) ABSTRACT

Provided herein are dispersible powder compositions comprising metal oxide particles and water, as well as methods of preparing dispersible powder compositions.

### CROSS REFERENCE TO RELATED APPLICATIONS

This application claims the benefit of priority to U.S. Provisional Patent Application 63/495,281, filed Apr. 10, 2023, the entire contents of which are incorporated herein by reference.

### BACKGROUND

Zinc Oxide (ZnO) is a semiconductor with a wide range of uses and applications (for example, manufacturing of rubbers, fuel cells, plastics, ceramics, LEDs, medications, and cosmetics). ZnO powders are produced via two major methods (American and French) that are energy consuming and require harsh conditions of temperature and pressure. The American process (aka the direct process) involves the reduction of zinc ore by heating with coal (such as anthracite), followed by the oxidation of zinc vapor in the same reactor, in a single production cycle. In the French process (aka the indirect process), first, the zinc must be processed into a metallic form, then the zinc metal is melted in a furnace and vaporized above 910 °C, typically setting furnace temperatures at 1000 °C. The immediate reaction of the zinc vapor with oxygen from the air produces ZnO. Several wet chemical processes to yield ZnO powders and other metal oxides have also been developed but they rely on organic solvents. See Kolodziejczak-Radzimska, A., et al. Materials, 7(4), pp. 2833-2881. As such, there exists a need for low-temperature wet chemical processes for manufacturing metal oxide compositions that do not involve organic solvents.

### SUMMARY OF THE INVENTION

In certain aspects, the present disclosure provides dispersible powder compositions, comprising:
metal oxide particles; and
water molecules;
wherein:
   a portion of the water molecules are associated with the surface of the metal oxide particles.

In further aspects, provided herein are methods for preparing a dispersible powder composition, the composition comprising:
metal oxide particles; and
water molecules;
wherein:
a portion of the water molecules are associated with the surface of the metal oxide particles; the method comprising:
dissolving at least one metal precursor in a first aqueous solution to afford a metal precursor solution;
adding the metal precursor solution to a buffer solution comprising water and a buffer, thereby affording a combined solution;
mixing the combined solution thereby causing the dispersible powder composition to precipitate from the combined solution; and
optionally separating the dispersible powder composition from the combined solution;
wherein the difference in temperature between the buffer solution and the metal precursor solution is from about 13 °C to about 78 °C.

### BRIEF DESCRIPTION OF THE DRAWINGS

FIG. 1 is a plot showing percent abundance as a function of hydrodynamic diameter in an exemplary sample of a ZnO composition of the disclosure, as measured using Dyanmic Light Scattering measurements.

FIG. 2 shows a schematic representation of an exemplary process for preparing ZnO compositions.

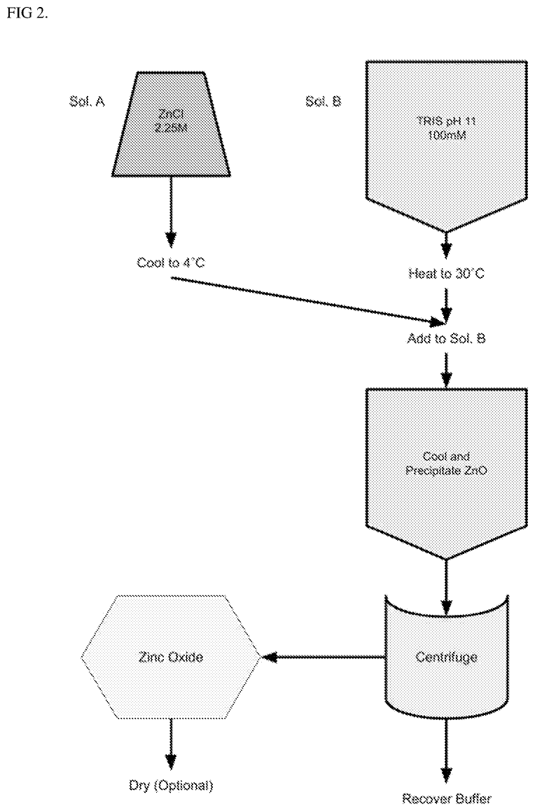

FIG. 3 shows a schematic representation of an exemplary process for preparing ZnO compositions comprising a dopant (Fe in this representation).

FIG. 4 shows various Fe-doped Zn compositions prepared using the process shown in FIG. 3, using varying amounts of FeCl₂ (lighter gray indicates lower iron content, darker gray indicates higher iron content).

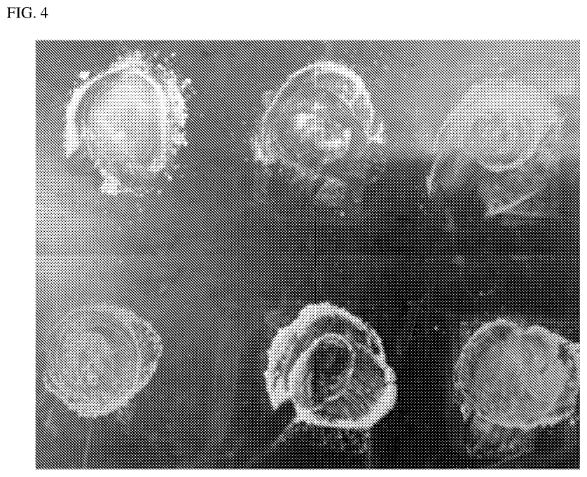

FIG. 5 is an image of ZnO particles obtained via Scanning Electron Microscopy (SEM). The image demonstrates the rough-textured surface topology of the particles, which enhances water association with the surface and leads to particles having a higher surface area than a smooth-textured spherical particle of the same size.

FIG. 6. is an image of ZnO particles obtained via Scanning Electron Microscopy (SEM).

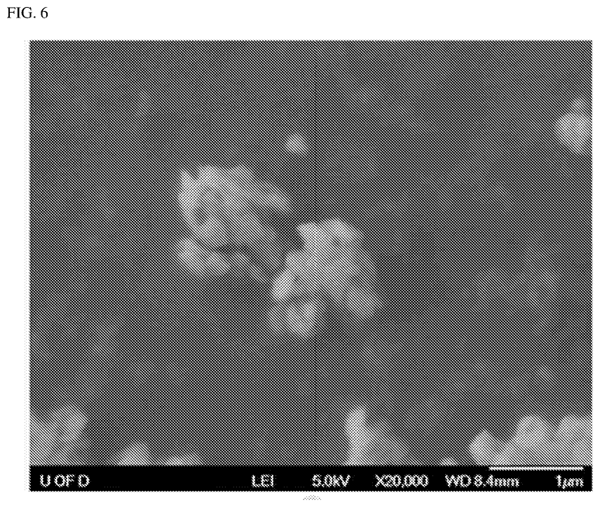

FIG. 7 is an image of ZnO particles obtained via Transmission Electron Microscopy (TEM). The image corroborates particle size data obtained via Dynamic Light Scattering (DLS) experiments and demonstrates an overall regular, substantially rounded geometry of the particles.

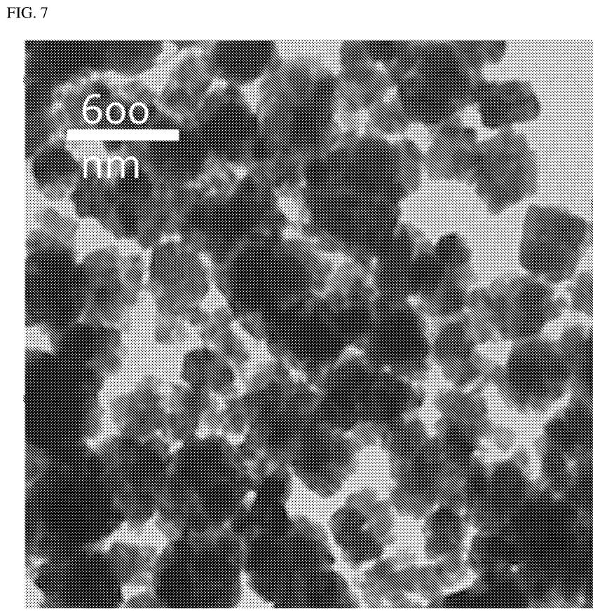

### DETAILED DESCRIPTION OF THE INVENTION

### Zinc Oxide

Zinc oxide is widely used in skincare and cosmetic products. It serves as a sunblock in sunscreen creams and lotions due to its ability to block both UVA and UVB rays. It's also found in diaper rash creams, calamine lotion, and makeup for its soothing, anti-inflammatory, and antimicrobial properties. It is often used as an additive in cosmetics for example tinted zinc oxide is used where color is desirable (sterile lipsticks and make-up of given colors—as conventional products can often incubate bacteria/fungi)

In the medical field, zinc oxide is used in ointments, creams, and lotions to treat a variety of skin conditions, including eczema, dermatitis, and burns. It promotes wound healing and prevents bacterial infections. Some examples of aqueous ZnO as an Antibacterial Agent include:
1. Aqueous based spray for prevention of bacterial aggregation on walls, sound-proof panels and any rough surface that need to be as sterile as possible
2. Aqueous based sprays as potential replacement for 70% ethanol solutions in tissue culture areas (70% ethanol solutions only have anti-bacterial/viral properties for less than a minute before dissipating).

Zinc oxide can be employed for a vast array of medical applications including:
1. Antibacterial coatings and sprays for
   a. surgery equipment
   b. medical clothing
2. Dedicated bactericidal agents against MRSA and other nosocomial agents
3. Skin protective agent (including diaper cream)
4. Anti-acne topical creams (using silver doping)
5. Cancer cell cytotoxicity inducing agents (cells with higher proliferation rates being most affected)
6. ZnO quantum dots used for targeted cancer therapy as drug delivery system
7. Tumor imaging and early detection of cancer biomarkers
8. Sensitivity enhancers for bacterial detection systems

Zinc oxide is a crucial ingredient in the rubber industry, where it acts as a catalyst in the vulcanization process, which is the chemical process for converting rubber into more durable materials. It enhances the rubber's resistance to heat, UV light, and other environmental factors.

In ceramic manufacturing, zinc oxide is used as a flux. It helps in the melting process and improves the quality of ceramic glazes, giving them strength, stability, and a brilliant finish.

Zinc oxide is utilized in paints and coatings for its ability to resist mold growth, provide UV protection, and improve adhesion and color retention properties. It's used in marine and industrial coatings for its anticorrosive properties. It can also be employed in water repellant and self-cleaning textile coatings.

In agriculture, zinc oxide is used as a micronutrient in fertilizers and animal feed. It is essential for crop growth and animal health, playing a significant role in DNA synthesis and metabolic processes.

Zinc oxide has semiconductor properties, making it useful in electronic materials and devices. It's used in varistors, ferrites, and as a transparent conducting oxide in liquid crystal displays (LCDs) and solar cells. Furthermore it is more efficient semi conductive materials for dye sensitive solar cells, it may enhance the sensitivity of gas sensors and it is employed in various field emission display (FED) applications

Zinc Oxide is used in batteries in a pure form, and also doped with materials like Lithium. Precise blends of oxides facilitate battery chemistry.

Zinc oxide is also used in environmental protection, acting as a photocatalyst in the degradation of pollutants and in water treatment processes to remove harmful substances.

Because of its added Antifungal properties, Zinc oxide can be employed for packaging: zinc oxide is also used in the producing and packing meat products or fish

The diverse applications of zinc oxide stem from its chemical and physical properties, making it an indispensable material in modern technology and industry.

The methods of synthesizing ZnO materials described herein provide a more environmentally friendly production process. These processes can utilize a metal aquo-complex mediated mineral synthesis to generate oxide particles. The process yields particles that are size-tunable and can be synthesized with a narrow size distribution including conditions to yield entirely non-nano products and suitable for manufacturing of non-toxic products (for example, cosmetics and sunscreen). These particulates can have a narrow distribution of size. This process yields relatively uniform ZnO structures employing merely water and two aqueous reactants (i.e. salts and biocompatible buffers). The reaction may occur rapidly (e.g., instantaneously) and the process of recovery of material can be fast. One additional feature is that it is possible, with this methodology, to synthesize ZnO particulates with a self-similar fractal-like spherical morphology. This structure can latch onto bacteria showing enhanced bacteriostatic/bactericidal properties that ZnO has been proven to have. This self-similar structure can enhance the water coordination in aqueous environments, and also potentially yield a larger surface area to mass ratio that is optimal for catalytic applications of ZnO.

### Iron Oxide

Iron oxides are a group of chemical compounds composed of iron and oxygen. They come in various forms, including magnetite (Fe3O4), hematite (Fe2O3), and goethite (FeO(OH)), among others. These compounds are widely used across different industries due to their pigmentation properties and as catalysts. Here's a brief overview of their uses across different sectors. There are naturally occurring deposits of iron oxides, but there are many applications that require a precisely made synthetic iron oxide.

The most common use of iron oxides is as pigments in paints, coatings, concrete colorings, and plastics. They provide a wide range of colors, from yellow to red, brown, and black, offering natural, non-toxic color options for manufacturers. Their stability against sunlight and atmospheric conditions makes them ideal for outdoor applications.

In the construction industry, iron oxide is used to color concrete, mortar, and pavers. The pigments can produce a wide range of earthy tones, enhancing the aesthetic appeal of architectural elements without compromising the material's strength or durability.

Iron oxides are used in cosmetics, particularly in foundations, powders, and lipsticks, to provide color variations. They are favored for their non-toxicity and inertness, making them safe for use on skin.

Iron oxides are used as catalysts in various chemical reactions, including the Haber-Bosch process for ammonia synthesis and in the Fischer-Tropsch process for producing liquid hydrocarbons from carbon monoxide and hydrogen. Their catalytic properties help in the efficient production of chemicals and fuels.

Magnetite (Fe3O4) is used in magnetic storage media, including tapes and hard disk drives, due to its magnetic properties. It enables the recording and storage of data through the alignment of its magnetic particles.

Iron oxides are utilized in water treatment processes for removing arsenic and other heavy metals from water. Their adsorptive properties help in trapping contaminants, making water safe for consumption and use.

Due to their rich colors and stability, iron oxides have been used historically as pigments for artwork, pottery glazes, and decorative finishes. They continue to be popular in artisanal and craft applications.

Iron oxides' versatility and non-toxic nature make them valuable in a wide array of applications, from industrial manufacturing to environmental management and art.

### Magnesium Oxide

Magnesium oxide (MgO), often known as magnesia, is a versatile mineral with a wide range of industrial, agricultural, environmental, and health applications. Here's a brief overview of its uses across different sectors:

One of the primary uses of magnesium oxide is in the production of refractory bricks and shapes used to line furnaces, kilns, incinerators, and reactors. MgO's high melting point (around 2,800 °C or 5,072 °F) and ability to withstand extreme temperatures without degrading make it ideal for these applications, particularly in the steel and cement industries.

Magnesium oxide is used in water treatment, flue gas desulfurization, and environmental remediation. It acts as a neutralizing agent to treat acidic wastewaters and soils, and as an adsorbent to capture heavy metals and pollutants. In flue gas treatments, MgO helps remove sulfur dioxide from exhaust gases, reducing air pollution.

In agriculture, magnesium oxide serves as a magnesium supplement in animal feeds and fertilizers. Magnesium is an essential nutrient for plant and animal health, playing a key role in chlorophyll formation and enzyme activation.

Magnesium oxide is widely used in the pharmaceutical industry as a dietary supplement for magnesium, an antacid for treating heartburn and indigestion, and a laxative. Its health applications leverage its ability to neutralize stomach acid and its laxative properties.

MgO is used in the construction industry as a component of magnesium oxychloride cement (Sorel cement), which is used for fireproofing, flooring, and wallboards. It offers fire resistance, moisture resistance, and mold resistance in building materials.

In ceramics and glass manufacturing, magnesium oxide acts as a flux to lower the melting point of raw materials, improving the properties of ceramic products and glass, enhancing their thermal and mechanical properties.

MgO is used as a catalyst and in the production of various chemicals, including magnesium salts, and as a raw material in the manufacture of magnesium metal through thermal reduction processes.

Magnesium oxide is employed in the pulp and paper industry as a bleaching agent in the deinking process of recycled paper and as a clarifier to clean process water, improving the quality and brightness of the paper.

In food production, MgO is used as an anti-caking agent, pH regulator, and nutrient supplement in some food products, ensuring quality and safety.

Along with calcium oxide, Magnesium oxide will capture and convert carbon dioxide into Magnesium Carbonate, sequestering the CO₂ in the process. This is used in applications of MgO weathering for CO₂ drawdown, as well as industrial emissions reduction.

Magnesium oxide's diverse applications are attributed to its chemical properties, including its reactivity, thermal stability, and ability to neutralize acids, making it a valuable material across a broad spectrum of industries.

### Manganese Oxide

Manganese oxide comes in several forms, with manganese(IV) oxide (MnO2) being the most common. It's used in various industries due to its catalytic, pigmentation, and electrochemical properties. Here's a brief overview of its uses across different sectors:

Manganese oxide is a key component in the manufacture of dry cell batteries, such as alkaline batteries and zinc-carbon batteries. MnO2 acts as a depolarizer, converting the hydrogen gas generated within the battery to water, thus preventing the battery from swelling and leaking.

Manganese oxides are used as catalysts in chemical reactions, including the oxidation of volatile organic compounds (VOCs) and the synthesis of fine chemicals. Their catalytic activity is also crucial in environmental applications, such as in the catalytic conversion of airborne pollutants in automotive exhaust systems.

In water treatment processes, manganese oxide is used for its oxidative properties to remove iron, manganese, and other contaminants from drinking water, making it safer for consumption.

Manganese oxides serve as pigments in ceramics, paints, and varnishes, offering colors ranging from brown to black. They are used in traditional pottery and to give glass a purple color.

In the steel industry, manganese oxide is used as an oxidizing agent in steel production to remove sulfur and oxygen impurities from the molten metal. It improves the hardness and strength of steel while preventing the formation of harmful iron sulfides.

Beyond batteries, manganese oxide is explored for its use in supercapacitors and as a cathode material in lithium-ion batteries due to its high energy density and ability to undergo reversible redox reactions.

Manganese oxide is used as a micronutrient in animal feed and fertilizers to correct manganese deficiencies in plants and animals, which is essential for various biological processes.

MnO2 is employed in the remediation of contaminated soils and groundwater. Its strong oxidative properties allow it to break down pollutants, including chlorinated solvents and dyes, making it useful in cleaning up environmental hazards.

Manganese oxide's versatility stems from its chemical properties, which make it invaluable in energy storage, environmental protection, and materials science, among other applications.

### Vanadium Oxide

Vanadium oxide, particularly vanadium(V) oxide (V2O5), is a significant industrial compound with various applications, primarily due to its oxidative, catalytic, and electrical properties. Here's a brief overview of its uses across different sectors:

The most significant industrial use of vanadium(V) oxide is as a catalyst in the Contact Process for the production of sulfuric acid (H2SO4). V2O5 enables the oxidation of sulfur dioxide (SO2) to sulfur trioxide (SO3), which is then converted to sulfuric acid, a critical industrial chemical used in numerous processes.

Vanadium oxides are used in the steel industry to produce high-strength steel alloys. Adding vanadium to steel significantly enhances its strength, toughness, and resistance to wear and corrosion. This is essential for applications in construction, automotive components, and tools.

In ceramics, vanadium oxide is used as a coloring agent and for its properties that influence the electrical conductivity and thermal resistance of ceramic materials. It can produce a variety of colors, from green to blue, in glazes and glass.

Vanadium(V) oxide is used in the manufacture of vanadium redox flow batteries (VRFBs), a type of rechargeable flow battery that takes advantage of vanadium ions in different oxidation states to store chemical potential energy. VRFBs are used for energy storage applications, such as storing wind and solar energy, due to their scalability, long lifespan, and ability to release large amounts of stored energy quickly.

Due to its ability to change oxidation states, vanadium oxide is used in the development of sensors for detecting gasses and environmental pollutants. It is particularly useful in sensors that detect changes in electrical resistance when exposed to specific chemicals.

Vanadium oxides are used in thin-film coatings for optical devices, where they contribute to controlling light transmission and reflection. They are also explored for their potential in electronic applications, such as in the development of phase change memory devices and as components in field-effect transistors (FETs).

Vanadium oxide can catalyze the oxidation of pollutants, including the removal of nitrogen oxides (NOx) from industrial flue gases, contributing to air pollution control efforts.

In the R&D sector, vanadium oxide's unique properties are being explored for use in smart windows that can change transparency in response to temperature, improving energy efficiency in buildings.

Vanadium oxide's diverse applications are a testament to its importance in modern industrial processes, materials science, and energy technology, leveraging its unique chemical and physical properties.

### Copper Oxide

Copper oxide exists in two common forms: cupric oxide (CuO) and cuprous oxide (Cu2O), each with distinct properties and applications across various industries. Here's a brief overview of how copper oxide is utilized:

Copper oxide is used in the production of semiconductor devices due to its unique electrical properties. Cuprous oxide (Cu2O) is a p-type semiconductor material used in photovoltaic cells, photodetector devices, and in the creation of conductive films.

Both forms of copper oxide serve as catalysts in chemical reactions. They are used in the synthesis of fine chemicals, in the catalytic reduction of pollutants, and in the production of methanol and synthetic fuels. CuO, in particular, is used as a catalyst in the hydrogenation of carbon monoxide in the Fischer-Tropsch process.

Copper oxides are used as pigments in ceramics, glass, and paints. Cu2O gives a distinctive red color, while CuO produces greens and blacks. These oxides are valued for their stability and vibrancy in various artistic and industrial applications.

Copper oxide is an active ingredient in antifouling paints used on ships and marine infrastructure. These paints prevent the growth of barnacles and other marine organisms on hulls, thereby improving ships' efficiency and durability.

Cupric oxide is used in agriculture as a component in fungicides, bactericides, and as a nutrient supplement in animal feed and fertilizers. Its antimicrobial properties help protect crops from fungal and bacterial diseases.

Copper oxide is investigated for use in battery technology, particularly in lithium-ion batteries, as an electrode material. It offers the potential for high capacity and stability in energy storage applications.

In glass and ceramics, copper oxide is used to create color and to control optical properties. It can produce a range of colors from deep red to green, depending on the concentration and the form used.

Copper oxide nanoparticles are explored for their use in environmental cleanup, such as in the degradation of pollutants and in water treatment processes. Their high surface area and reactivity make them effective in capturing and breaking down harmful substances.

Copper oxides are incorporated into textiles and surface coatings for their antimicrobial properties. This application is particularly valuable in healthcare settings, where reducing the spread of bacteria and viruses is crucial.

Copper oxide's versatility, stemming from its electrical, catalytic, and antimicrobial properties, makes it a valuable material in a wide array of applications, from advanced technology to environmental management and beyond.

### Dispersible Powder Compositions

In certain aspects, the present disclosure provides dispersible powder compositions, comprising:
metal oxide particles; and
water molecules;
wherein:
   a portion of the water molecules are associated with the surface of the metal oxide particles.

In certain embodiments, provided herein are dispersible powder compositions comprising:
metal oxide particles; and
water molecules;
wherein:
the water molecules comprise about 10 wt % to about 80 wt % of the composition; and
a portion of the water molecules are associated with the surface of the metal oxide particles.

In certain embodiments, provided herein are dispersible powder compositions comprising:
metal oxide particles; and
water molecules;
wherein:
a portion of the water molecules are associated with the surface of the metal oxide particles; and
the metal oxide particles have a hydrodynamic diameter from about 15 nm to about 1500 nm.

In certain embodiments, provided herein are dispersible powder compositions comprising:
metal oxide particles; and
water molecules;
wherein:
a portion of the water molecules are associated with the surface of the metal oxide particles; and
the metal oxide particles have a diameter from about 15 nm to about 1300 nm.

In certain embodiments, provided herein are dispersible powder compositions comprising:
metal oxide particles; and
water molecules;
wherein:
a portion of the water molecules are associated with the surface of the metal oxide particles; and
the metal oxide particles have a diameter distribution characterized by a D90 of less than about 1200 nm.

In certain embodiments, provided herein are dispersible powder compositions comprising:
metal oxide particles; and
water molecules;
wherein:
a portion of the water molecules are associated with the surface of the metal oxide particles; and
the metal oxide particles have a diameter distribution characterized by a D90 of less than about 1200 nm.

In certain embodiments, provided herein are dispersible powder compositions comprising:
metal oxide particles; and
water molecules;
wherein:
a portion of the water molecules are associated with the surface of the metal oxide particles; and
the metal oxide particles have a hydrodynamic diameter distribution characterized by a D90 of less than about 1300 nm.

In certain embodiments, compositions of the present disclosure comprise water molecules that are “associated” with the surface of the metal oxide particles. As will be appreciated by one of ordinary skill in the art, these water molecules may be associated with the surface of the metal oxide particles through chemical bonding interactions, including but not limited to: dative bonds, electrostatic (e.g., ionic) bonds, covalent bonds, or hydrogen bonds. These interactions are sufficient to maintain high water content in the dispersible powder compositions, but also do not necessarily prevent the removal of a portion said water molecules by, e.g., vacuum drying or desiccation, as may desirable. These associated water molecules confer advantageous properties to the dispersible powder compositions described herein, over other compositions known in the art. These advantages include, for example: higher total water content of the composition while still maintaining a powder-like form (as opposed to a slurry), improved flocculation and aggregate formation, and improved dispersibility in aqueous mixtures.

In certain embodiments, the metal oxide particles comprise a metal selected from a Group 1 metal, Group 2 metal, Group 3 metal, Group 5 metal, Group 6 metal, Group 7 metal, Group 8 metal, Group 11 metal, and Group 12 metal. In further embodiments, the metal oxide particles comprise a metal selected from a Group 1 metal. In yet further embodiments, the metal oxide particles comprise a metal selected from a Group 2 metal. In still further embodiments, the metal oxide particles comprise a metal selected from a Group 3 metal. In further embodiments, the metal oxide particles comprise a metal selected from Group a 5 metal. In yet further embodiments, the metal oxide particles comprise a metal selected from a Group 6 metal. In still further embodiments, the metal oxide particles comprise a metal selected from a Group 7 metal. In further embodiments, the metal oxide particles comprise a metal selected from a Group 8 metal. In yet further embodiments, the metal oxide particles comprise a metal selected from a Group 11 metal. In still further embodiments, the metal oxide particles comprise a metal selected from a Group 12 metal.

In certain embodiments, the metal oxide particles comprise Li, Mg, Ca, Eu, V, Cr, Mn, Fe, Zn, Ag, or a combination thereof. In further embodiments, the metal oxide particles comprise Li. In yet further embodiments, the metal oxide particles comprise Mg. In still further embodiments, the metal oxide particles comprise Ca. In further embodiments, the metal oxide particles comprise Eu. In yet further embodiments, the metal oxide particles comprise V. In still further embodiments, the metal oxide particles comprise Cr. In further embodiments, the metal oxide particles comprise Mn. In yet further embodiments, the metal oxide particles comprise Fe. In still further embodiments, the metal oxide particles comprise Zn. In further embodiments, the metal oxide particles comprise Ag. In certain embodiments, the metal oxide particles do not comprise Ba.

In certain embodiments, the metal oxide particles comprise at least two metallic elements. In further embodiments, the at least two metallic elements are each independently selected from a Group 1 metal, Group 2 metal, Group 3 metal, Group 5 metal, Group 6 metal, Group 7 metal, Group 8 metal, Group 11 metal, and Group 12 metal. In yet further embodiments, the at least two metallic elements are each independently selected from a Group 8 metal and a Group 12 metal. In still further embodiments, the at least two metallic elements are each independently selected from a Group 11 metal or a Group 12 metal. In further embodiments, the at least two metallic elements are each independently selected from Li, Mg, Ca, Eu, V, Cr, Mn, Fe, Zn, and Ag. In yet further embodiments, the at least two metallic elements are each independently selected from Fe, Zn, and Ag. In certain embodiments, the at least two metallic elements are Fe and Zn. In further embodiments, the at least two metallic elements are Fe and Ag. In yet further embodiments, the at least two metallic elements are Fe, Ag, and Zn. In still further embodiments, the at least two metallic elements are Ag and Zn.

In certain embodiments, the metal oxide particles further comprise at least one dopant, and the dopant is a monoatomic cation. In further embodiments, the at least one dopant is a metallic element. In yet further embodiments, the at least one dopant is a semi-metallic element. In still further embodiments, the at least one dopant is a nonmetallic element. In further embodiments, the at least one dopant is iron. In yet further embodiments, the at least one dopant is silver. In still further embodiments, the at least one dopant is lithium. In further embodiments, the at least one dopant is a combination of iron and silver.

In certain embodiments, the at least one dopant is present in an amount of from about 0.01 wt % to about 50 wt % relative to a total weight of the dispersible powder composition. In further embodiments, the at least one dopant is present in an amount of from about 0.01 wt % to about 0.05 wt %. In yet further embodiments, the at least one dopant is present in an amount of from about 0.06 wt % to about 0.1 wt %. In still further embodiments, the at least one dopant is present in an amount of from about 0.11 wt % to about 1 wt %. In further embodiments, the at least one dopant is present in an amount of from about 1.1 wt % to about 5 wt %. In yet further embodiments, the at least one dopant is present in an amount of from about 5.1 wt % to about 15 wt %. In still further embodiments, the at least one dopant is present in an amount of from about 15.1 wt % to about 35 wt %. In further embodiments, the at least one dopant is present in an amount of from about 35.1 wt % to about 50 wt %. In certain embodiments, the at least one dopant is present in an amount of about 0.2 wt %, about 0.5 wt %, about 0.75 wt %, about 1 wt %, about 2 wt %, about 5 wt %, about 7.5 wt %, or about 10 wt %. In further embodiments, the at least one dopant is present in an amount of about 0.2 wt %. In yet further embodiments, the at least one dopant is present in an amount of about 0.5 wt %. In still further embodiments, the at least one dopant is present in an amount of about 0.75 wt %. In further embodiments, the at least one dopant is present in an amount of about 1 wt %. In yet further embodiments, the at least one dopant is present in an amount of about 2 wt %. In still further embodiments, the at least one dopant is present in an amount of about 5 wt %. In further embodiments, the at least one dopant is present in an amount of about 7.5 wt %. In yet further embodiments, the at least one dopant is present in an amount of about 10 wt %.

In certain embodiments, the dispersible powder composition does not comprise carbon. In some embodiments, the dispersible powder composition does not comprise silicon.

In certain embodiments, the metal oxide particles do not comprise a coating. In further embodiments, the metal oxide particles do not comprise a hydrophobic coating. In yet further embodiments, the metal oxide particles do not comprise a hydrophilic coating. In still further embodiments, the metal oxide particles do not comprise a coating comprising an organic dispersant. In further embodiments, the dispersible powder composition does not comprise an organic dispersant.

In certain embodiments, only the surface-associated water molecules are associated with the surface of the metal oxide particles. In further embodiments, the surface of the metal oxide particles has not been functionalized or modified by the addition of functional groups. In yet further embodiments, the functional groups comprise alkyl, alkenyl, alkynyl, alkoxy, siloxy, silyl, carboxyl, amino, or phosphoalkyl, or a combination thereof.

In certain embodiments, the metal oxide particles are in the form of a non-spherical aggregate having an increased surface area relative to a sphere of the same diameter. As will be appreciated by one of ordinary skill in the art, the increased surface area afforded by the topology and morphology of the metal oxide particles affords an increased number of sites at which water molecules may associate with the particles. This increased association results in compositions comprising a higher number of associated water molecules per particle than similarly-sized particles with a lower surface area (e.g., flatter surface topology). In certain embodiments, the metal oxide particles are in the form of a rounded, cauliflower-like shape.

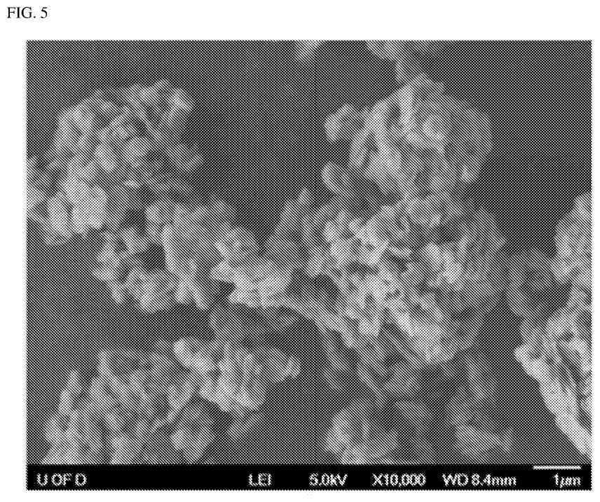

In certain embodiments, the metal oxide particles have a form substantially as shown in FIG. 5.

In certain embodiments, the dispersible powder composition has a maximum optical absorption wavelength (λₘₐₓ) at about 360 nm to about 390 nm. In further embodiments, the dispersible powder composition has a maximum optical absorption wavelength (λₘₐₓ) at about 365 nm to about 380 nm. In yet further embodiments, the dispersible powder composition has a maximum optical absorption wavelength (λₘₐₓ) at about 365 nm. In still further embodiments, the dispersible powder composition has a maximum optical absorption wavelength (λₘₐₓ) at about 370 nm. In further embodiments, the dispersible powder composition has a maximum optical absorption wavelength (λₘₐₓ) at about 375 nm. In yet further embodiments, the dispersible powder composition has a maximum optical absorption wavelength (λₘₐₓ) at about 380 nm. In certain embodiments, the dispersible powder composition has a maximum optical absorption wavelength (λₘₐₓ) at about 375 nm, with a molar absorption coefficient(s) of about 5×10⁻⁴ M⁻¹·cm⁻¹.

In certain embodiments, the water molecules comprise about 10 wt % to about 80 wt % of the composition. In further embodiments, the water molecules comprise about 10 wt % to about 20 wt % of the composition. In yet further embodiments, the water molecules comprise about 21 wt % to about 30 wt % of the composition. In still further embodiments, the water molecules comprise about 31 wt % to about 40 wt % of the composition. In further embodiments, the water molecules comprise about 41 wt % to about 50 wt % of the composition. In yet further embodiments, the water molecules comprise about 51 wt % to about 60 wt % of the composition. In still further embodiments, the water molecules comprise about 61 wt % to about 70 wt % of the composition. In further embodiments, the water molecules comprise about 71 wt % to about 80 wt % of the composition. In further embodiments, the water molecules comprise about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, or about 80 wt % of the composition.

As will be appreciated by one of ordinary skill in the art, any suitable means of measuring hydrodynamic diameters of metal oxide particles in solution may be used, and the exemplary DLS procedure set forth in the examples below demonstrates certain suitable procedures for obtaining these data. In certain embodiments, the hydrodynamic diameter is measured using Dynamic Light Scattering or Photon Correlation Spectroscopy.

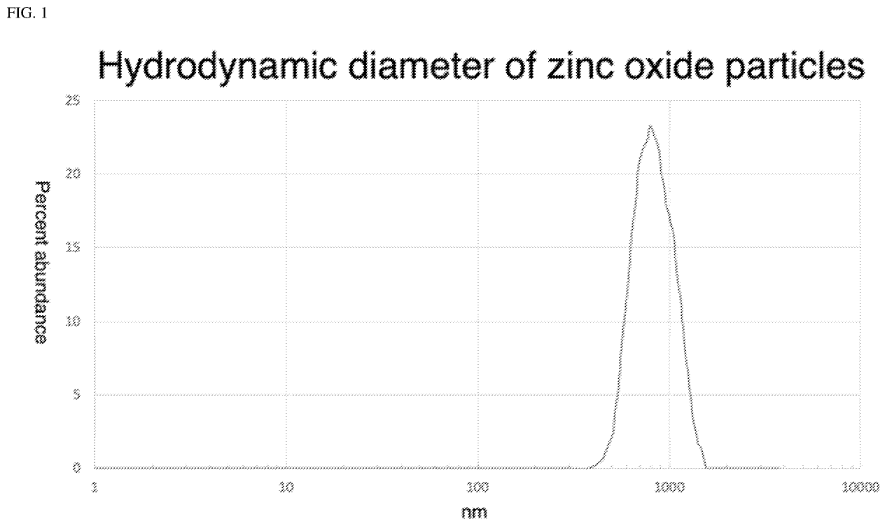

In certain embodiments, the metal oxide particles have a hydrodynamic diameter from about 15 nm to about 1500 nm. In further embodiments, the metal oxide particles have a hydrodynamic diameter from about 25 nm to about 1500 nm. In yet further embodiments, the metal oxide particles have a hydrodynamic diameter from about 50 nm to about 1500 nm. In still further embodiments, the metal oxide particles have a hydrodynamic diameter from about 75 nm to about 1500 nm. In further embodiments, the metal oxide particles have a hydrodynamic diameter from about 100 nm to about 1500 nm. In yet further embodiments, the metal oxide particles have a hydrodynamic diameter of from about 150 nm to about 1500 nm. In still further embodiments, the metal oxide particles have a hydrodynamic diameter of from about 200 nm to about 1500 nm. In further embodiments, the metal oxide particles have a hydrodynamic diameter of from about 250 nm to about 1500 nm. In still further embodiments, the metal oxide particles have a hydrodynamic diameter of from about 300 nm to about 1500 nm. In yet further embodiments, the metal oxide particles have a hydrodynamic diameter of from about 150 nm to about 1450 nm. In still further embodiments, the metal oxide particles have a hydrodynamic diameter of from about 150 to about 1400 nm. In further embodiments, the metal oxide particles have a hydrodynamic diameter of from about 150 nm to about 1350 nm. In further embodiments, the metal oxide particles have a hydrodynamic diameter of from about 150 nm to about 1300 nm. In yet further embodiments, the metal oxide particles have a hydrodynamic diameter of from about 150 nm to about 1250 nm. In still further embodiments, the metal oxide particles have a hydrodynamic diameter of from about 150 to about 1200 nm. In further embodiments, the metal oxide particles have a hydrodynamic diameter of from about 300 to about 1150 nm. In certain embodiments, the metal oxide particles have a hydrodynamic diameter distribution substantially as shown in FIG. 1.

As will be appreciated by one of ordinary skill in the art, any suitable means of measuring diameters of metal oxide particles may be used, and the exemplary TEM procedure set forth in the examples below demonstrates certain suitable procedures for obtaining these data. In certain embodiments, the diameter is measured by Transmission Electron Microscopy (TEM).

In certain embodiments, the metal oxide particles have a diameter from about 15 nm to about 1300 nm. In further embodiments, the metal oxide particles have a diameter from about 25 nm to about 1300 nm. In yet further embodiments, the metal oxide particles have a diameter from about 50 nm to about 1300 nm. In still further embodiments, the metal oxide particles have a diameter from about 75 nm to about 1300 nm. In further embodiments, the metal oxide particles have a diameter from about 100 nm to about 1300 nm. In yet further embodiments, the metal oxide particles have a diameter from about 125 nm to about 1300 nm. In still further embodiments, the metal oxide particles have a diameter from about 150 nm to about 1300 nm. In further embodiments, the metal oxide particles have a diameter from about 175 nm to about 1300 nm. In yet further embodiments, the metal oxide particles have a diameter from about 200 nm to about 1300 nm. In still further embodiments, the metal oxide particles have a diameter from about 225 nm to about 1300 nm. In further embodiments, the metal oxide particles have a diameter from about 50 nm to about 1275 nm. In yet further embodiments, the metal oxide particles have a diameter from about 50 nm to about 1250 nm. In still further embodiments, the metal oxide particles have a diameter from about 50 nm to about 1225 nm. In further embodiments, the metal oxide particles have a diameter from about 50 nm to about 1200 nm. In yet further embodiments, the metal oxide particles have a diameter from about 50 nm to about 1175 nm. In still further embodiments, the metal oxide particles have a diameter from about 50 nm to about 1150 nm. In further embodiments, the metal oxide particles have a diameter from about 50 nm to about 1125 nm. In yet further embodiments, the metal oxide particles have a diameter from about 50 nm to about 1100 nm. In still further embodiments, the metal oxide particles have a diameter from about 50 nm to about 1075 nm. In further embodiments, the metal oxide particles have a diameter from about 50 nm to about 1050 nm. In yet further embodiments, the metal oxide particles have a diameter from about 50 nm to about 1025 nm. In still further embodiments, the metal oxide particles have a diameter from about 50 nm to about 1000 nm. In further embodiments, the metal oxide particles have a diameter from about 50 nm to about 975 nm. In yet further embodiments, the metal oxide particles have a diameter from about 50 nm to about 950 nm. In still further embodiments, the metal oxide particles have a diameter from about 50 nm to about 925 nm. In further embodiments, the metal oxide particles have a diameter from about 100 nm to about 1250 nm. In yet further embodiments, the metal oxide particles have a diameter from about 200 nm to about 920 nm.

In certain embodiments, the metal oxide particles have a diameter distribution characterized by a D90 of less than about 1200 nm, less than about 1175 nm, less than about 1150 nm, less than about 1125 nm, less than about 1100 nm, less than about 1075 nm, or less than about 1050 nm. In further embodiments, the diameter distribution is characterized by a D90 from about 5 nm to about 1200 nm. In yet further embodiments, the diameter distribution is characterized by a D90 of about 1200 nm. In still further embodiments, the diameter distribution is characterized by a D90 of about 1175 nm. In further embodiments, the diameter distribution is characterized by a D90 of about 1150 nm. In yet further embodiments, the diameter distribution is characterized by a D90 of less than about 1125 nm.

In certain embodiments, the metal oxide particles have a diameter distribution characterized by a D50 of less than about 775 nm, less than about 750 nm, less than about 725 nm, less than about 700 nm, less than about 675 nm, less than about 650 nm, less than about 625 nm, less than about 600 nm, or less than about 575 nm. In further embodiments, the metal oxide particles have a diameter distribution characterized by a D50 of less than about 650 nm. In yet further embodiments, the metal oxide particles have a diameter distribution characterized by a D50 of about 650 nm. In still further embodiments, the diameter distribution is characterized by a D50 from about 5 nm to about 650 nm.

In certain embodiments, the metal oxide particles have a diameter distribution characterized by a D10 of less than about 45 nm, less than about 40 nm, less than about 35 nm, less than about 30 nm, less than about 25 nm, less than about 20 nm, or less than about 15 nm. In further embodiments, the diameter distribution is characterized by a D10 of less than about 25 nm. In yet further embodiments, the diameter distribution is characterized by a D10 of about 25 nm. In still further embodiments, the diameter distribution is characterized by a D10 from about 5 nm to about 25 nm.

In certain embodiments, the metal oxide particles have a hydrodynamic diameter distribution characterized by a D90 of less than about 1400 nm, less than about 1375 nm, less than about 1350 nm, less than about 1325 nm, less than about 1300 nm, less than about 1275 nm, less than about 1250 nm, less than about 1225 nm, or less than about 1200 nm. In further embodiments, the metal oxide particles have a hydrodynamic diameter distribution characterized by a D90 of less than about 1300 nm. In yet further embodiments, the metal oxide particles have a hydrodynamic diameter distribution characterized by a D90 of about 1300 nm. In still further embodiments, the hydrodynamic diameter distribution is characterized by a D90 from about 5 nm to about 1300 nm.

In certain embodiments, the metal oxide particles have a hydrodynamic diameter distribution characterized by a D50 of less than about 875 nm, less than about 850 nm, less than about 825 nm, less than about 800 nm, less than about 775 nm, less than about 750 nm, less than about 725 nm, less than about 700 nm, less than about 675 nm, or less than about 650 nm. In further embodiments, the metal oxide particles have a hydrodynamic diameter distribution characterized by a D50 of less than about 700 nm. In yet further embodiments, the metal oxide particles have a hydrodynamic diameter distribution characterized by a D50 of about 700 nm. In still further embodiments, the hydrodynamic diameter distribution is characterized by a D50 from about 5 nm to about 700 nm.

In certain embodiments, the metal oxide particles have a hydrodynamic diameter distribution characterized by a D10 of less than about 200 nm, less than about 175 nm, less than about 150 nm, less than about 125 nm, less than about 100 nm, less than about 75 nm, or less than about 50 nm. In further embodiments, the hydrodynamic diameter distribution is characterized by a D10 of less than about 150 nm. In yet further embodiments, the hydrodynamic diameter distribution is characterized by a D10 of about 150 nm. In still further embodiments, the hydrodynamic diameter distribution is characterized by a D10 from about 5 nm to about 150 nm.

In certain embodiments, provided herein are aqueous compositions comprising a dispersible powder composition as described herein and an aqueous carrier. In further embodiments, the aqueous carrier is a pharmaceutically acceptable carrier or a cosmetically acceptable carrier. In yet further embodiments, the aqueous carrier is selected from: water, saline solutions, biocompatible buffers, hydrogels or other gel bases, and micellar water. As will be appreciated by one of ordinary skill in the art, the aqueous composition may further comprise viscosity modifiers, emulsifiers, detergents, and/or other additives to achieve a desired combination of physical properties, depending on the desired application.

### Methods for Producing Dispersible Powder Compositions

In certain aspects, provided herein are methods for of preparing a composition of the disclosure, the method comprising:
dissolving at least one metal precursor in a first aqueous solution to afford a metal precursor solution;
adding the metal precursor solution to a buffer solution comprising water and a buffer, thereby affording a combined solution;
stirring the combined solution until the dispersible powder composition precipitates from the combined solution; and
optionally separating the dispersible powder composition from the combined solution;
wherein the difference in temperature between the buffer solution and the metal precursor solution is from about 13 °C to about 78 °C.

In some aspects, provided herein are methods for preparing a dispersible powder composition, the composition comprising:
metal oxide particles; and
water molecules;
wherein:
a portion of the water molecules are associated with the surface of the metal oxide particles;
the method comprising:
dissolving at least one metal precursor in a first aqueous solution to afford a metal precursor solution;
adding the metal precursor solution to a buffer solution comprising water and a buffer, thereby affording a combined solution;
mixing the combined solution thereby causing the dispersible powder composition to precipitate from the combined solution; and
optionally separating the dispersible powder composition from the combined solution;
wherein the difference in temperature between the buffer solution and the metal precursor solution is from about 13 °C to about 78 °C.

As will be appreciated by one of ordinary skill in the art, the difference in temperature between the buffer solution and the metal precursor solution (dT) must be sufficient in magnitude to allow the reaction(s) forming the metal oxide to proceed. Additionally, one of ordinary skill in the art would understand how to determine a suitable dT for conducting a method of the disclosure for any combination of metal precursor solution and buffer solutions described herein.

In certain embodiments, the difference in temperature between the buffer solution and the metal precursor solution is from about 15 °C to about 50 °C. In further embodiments, the difference in temperature is about 20 °C to about 40 °C. In yet further embodiments, the difference in temperature between the buffer solution and the metal precursor is from about 20 °C to about 30 °C. In still further embodiments, the difference in temperature between the buffer solution and the metal precursor is about 15 °C, about 20 °C, about 22 °C, about 24 °C, about 25 °C, about 26 °C, about 28 °C, about 30 °C, about 35 °C, about 40 °C, about 45 °C, or about 50 °C. In further embodiments, the difference in temperature between the buffer solution and the metal precursor is about 25 °C.

In certain embodiments, the temperature of the metal precursor solution is from about 40 °C to about 90 °C when added to the buffer solution. In further embodiments, the temperature of the metal precursor solution is about 60 °C to about 65 °C when added to the buffer solution.

In certain embodiments, the buffer solution is at a buffer solution temperature from about 12 °C to about 32 °C when the metal precursor solution is added. In further embodiments, the buffer solution is at a buffer solution temperature of about 22 °C to about 24 °C when the metal precursor solution is added.

In certain embodiments, the first aqueous solution comprises reverse osmosis deionized water.

In certain embodiments, the method comprises separating the dispersible powder composition from the combined solution.

In certain embodiments, each of the at least one metal precursors is a neutral ionic compound comprising a metal cation and at least one counter-anion. In some embodiments, the at least one metal precursor is one metal precursor. In other embodiments, the at least one metal precursor is at least two metal precursors, and each of the at least two metal precursors is a neutral ionic compound comprising a metal cation and at least one counter-anion. In further embodiments, the metal cation comprises a metal selected from a Group 1 metal, a Group 2 metal, a Group 3 metal, a Group 5 metal, a Group 6 metal, a Group 7 metal, a Group 8 metal, a Group 11 metal, and a Group 12 metal, or a combination thereof. In yet further embodiments, the metal cation comprises Li, Mg, Ca, Eu, V, Cr, Mn, Fe, Zn, Ag, or a combination thereof. In still further embodiments, the metal cation comprises Li. In further embodiments, the metal cation comprises Mg. In yet further embodiments, the metal cation comprises Ca. In still further embodiments, the metal cation comprises Eu. In further embodiments, the metal cation comprises V. In yet further embodiments, the metal cation comprises Cr. In still further embodiments, the metal cation comprises Mn. In further embodiments, the metal cation comprises Fe. In yet further embodiments, the metal cation comprises Zn. In still further embodiments, the metal cation comprises Ag. In certain embodiments, the metal cation is Zn²⁺. In some embodiments, the metal cation does not comprise Ba.

In certain embodiments, methods of the disclosure further comprise dissolving at least one dopant precursor in the first aqueous solution, wherein the at least one dopant precursor is a neutral ionic compound comprising at least one dopant and at least one counter-anion, and wherein the at least one dopant is a monoatomic cation. In further embodiments, the at least one dopant is a metallic element. In yet further embodiments, the at least one dopant is a semi-metallic element. In still further embodiments, the at least one dopant is a nonmetallic element. In further embodiments, the at least one dopant is iron. In yet further embodiments, the at least one dopant is silver. In still further embodiments, the at least one dopant is lithium. In further embodiments, the at least one dopant is iron and silver.

In certain embodiments, the at least one dopant is present in an amount of from about 0.01 wt % to about 50 wt % relative to a total weight of the dispersible powder composition. In further embodiments, the at least one dopant is present in an amount of from about 0.01 wt % to about 0.05 wt %. In yet further embodiments, the at least one dopant is present in an amount of from about 0.06 wt % to about 0.1 wt %. In still further embodiments, the at least one dopant is present in an amount of from about 0.11 wt % to about 1 wt %. In further embodiments, the at least one dopant is present in an amount of from about 1.1 wt % to about 5 wt %. In yet further embodiments, the at least one dopant is present in an amount of from about 5.1 wt % to about 15 wt %. In still further embodiments, the at least one dopant is present in an amount of from about 15.1 wt % to about 35 wt %. In further embodiments, the at least one dopant is present in an amount of from about 35.1 wt % to about 50 wt %. In certain embodiments, the at least one dopant is present in an amount of about 0.2 wt %, about 0.5 wt %, about 0.75 wt %, about 1 wt %, about 2 wt %, about 5 wt %, about 7.5 wt %, or about 10 wt %. In further embodiments, the at least one dopant is present in an amount of about 0.2 wt %. In yet further embodiments, the at least one dopant is present in an amount of about 0.5 wt %. In still further embodiments, the at least one dopant is present in an amount of about 0.75 wt %. In further embodiments, the at least one dopant is present in an amount of about 1 wt %. In yet further embodiments, the at least one dopant is present in an amount of about 2 wt %. In still further embodiments, the at least one dopant is present in an amount of about 5 wt %. In further embodiments, the at least one dopant is present in an amount of about 7.5 wt %. In yet further embodiments, the at least one dopant is present in an amount of about 10 wt %.

In certain embodiments, the at least one counter-anion is selected from halide, nitrate, and acetate. In further embodiments, the at least one counter-anion is halide. In yet further embodiments, the at least one counter-anion is nitrate. In still further embodiments, the at least one counter-anion is acetate. In further embodiments, the at least one counter-anion is chloride, bromide, or iodide. In yet further embodiments, the at least one counter-anion is chloride.

In certain embodiments, the neutral ionic compound is selected from: ZnCl₂, ZnBr₂, Zn(NO₃)₂, FeCl₂, FeCl₃, Fe(NO₃)₂, Fe(NO₃)₃, AgCl, Ag(NO₃), CrCl₂, Eu(NO₃)₃, Lil, LiCl, Li(NO₃), MgCl₂, MgBr₂, Mg(NO₃)₂, MnCl₂, MnBr₂, Mn(NO₃)₂, VCl₂, VBr₂, and V(NO₃)₂, or a hydrate thereof. In further embodiments, the neutral ionic compound is ZnCl₂. In yet further embodiments, the neutral ionic compound is ZnBr₂ or a hydrate thereof. In still further embodiments, the neutral ionic compound is Zn(NO₃)₂ or a hydrate thereof. In further embodiments, the neutral ionic compound is FeCl₂ or a hydrate thereof. In yet further embodiments, the neutral ionic compound is FeCl₃ or a hydrate thereof. In still further embodiments, the neutral ionic compound is Fe(NO₃)₂ or a hydrate thereof. In further embodiments, the neutral ionic compound is AgCl or a hydrate thereof. In yet further embodiments, the neutral ionic compound is Ag(NO₃) or a hydrate thereof. In still further embodiments, the neutral ionic compound is CrCl₂ or a hydrate thereof. In further embodiments, the neutral ionic compound is Eu(NO₃)₃ or a hydrate thereof. In yet further embodiments, the neutral ionic compound is LiI or a hydrate thereof. In still further embodiments, the neutral ionic compound is LiCl or a hydrate thereof. In further embodiments, the neutral ionic compound is Li(NO₃) or a hydrate thereof. In yet further embodiments, the neutral ionic compound is MgCl₂ or a hydrate thereof. In still further embodiments, the neutral ionic compound is MgBr₂ or a hydrate thereof. In further embodiments, the neutral ionic compound is Mn(NO₃)₂ or a hydrate thereof. In yet further embodiments, the neutral ionic compound is VCl₂ or a hydrate thereof. In still further embodiments, the neutral ionic compound is VBr₂ or a hydrate thereof. In further embodiments, the neutral ionic compound is V(NO₃)₂ or a hydrate thereof.

In certain embodiments, the at least one metal precursor is water-soluble. In further embodiments, the at least one metal precursor has a solubility in water of at least about 0.01 mol/L.

In certain embodiments, the at least one metal precursor is a hydrate.

In certain embodiments, each of the at least one metal precursors solution has a concentration from about 0.1 M to about 5 M. In further embodiments, the concentration of the metal precursor in the metal precursor solution is from about 0.5 M to about 1.5 M. In yet further embodiments, the concentration of the metal precursor in the metal precursor solution is about 0.5 M, about 0.75 M, about 1.0 M, about 1.25 M, or about 1.5 M.

In certain embodiments, the buffer solution has a pH greater than about 7.5. In further embodiments, the buffer solution has a pH greater than about 9. In still further embodiments, the buffer solution has a pH of about 7.5, about 8, about 8.5, about 9, about 9.5, about 10, about 10.5, about 11, about 11.5, or about 12. In further embodiments, the buffer solution has a pH of about 8.5. In yet further embodiments, the buffer solution has a pH of about 10. In still further embodiments, the buffer solution has a pH of about 11.

In certain embodiments, the water in the buffer solution comprises reverse osmosis deionized water.

In some embodiments, the buffer is non-toxic. In certain embodiments, the buffer is biocompatible.

In certain embodiments, the buffer is selected from: tris(hydroxymethyl)aminomethane (TRIS), borate (BO₃³⁻), TE (TRIS and EDTA), phosphates (e.g., PBS), and salts thereof. In further embodiments, the buffer is TRIS. In yet further embodiments, the buffer is BO₃³⁻.

In certain embodiments, the concentration of the buffer in the buffer solution is about 10 mM to 100 mM. In further embodiments, the concentration of the buffer in the buffer solution is about 10 mM, about 20 mM, about 30 mM, about 40 mM, about 50 mM, about 60 mM, about 70 mM, about 80 mM, about 90 mM, or about 100 mM.

In certain embodiments, the combined solution has a pH from about 7.5 to about 8.5 when the dispersible powder composition precipitates from the combined solution.

In certain embodiments, provided herein are dispersible powder compositions prepared by a method of the disclosure.

The disclosure includes exemplary process conditions (e.g., temperature, concentrations, etc.) which provide certain advantages in context of the systems and methods disclosed herein. However, any suitable conditions may be used, and the person of ordinary skill in the art will appreciate how to vary the conditions of any particular process described herein to obtain results and tune product distribution as needed for particular applications, as contemplated.

### Definitions

Unless otherwise defined herein, scientific and technical terms used in this application shall have the meanings that are commonly understood by those of ordinary skill in the art. Generally, nomenclature used in connection with, and techniques of, chemistry described herein, are those well-known and commonly used in the art.

The methods and techniques of the present disclosure are generally performed, unless otherwise indicated, according to conventional methods well known in the art and as described in various general and more specific references that are cited and discussed throughout this specification.

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

As used herein, the term “coating” is used to refer to one or more outer layers bound to the surface of a particle, the outer layer(s) comprising a plurality of molecules or chemical functional groups each connected to the surface of the molecule by ionic or covalent chemical bonds. A “coating” as defined herein does not include a plurality of water molecules associated with the surface of the particle, e.g., through hydrogen or dative bonding.

The term “hydrodynamic diameter” as used herein refers to the diameter of a theoretical perfectly solid spheroid particle that would exhibit the same hydrodynamic friction as the real metal oxide particle in the sample. As one of ordinary skill in the art would appreciate, experimentally determined hydrodynamic diameter values are slightly larger than the diameter values measured by optical techniques, e.g., TEM or SEM, because of the effects of solvent interactions with the particles as they diffuse through solution. For a detailed discussion of measurement of particle sizes, see, e.g., Sci Technol Adv Mater. 2018; 19, 732-745.

The term “diameter” as used herein refers to the diameter of a particle in a “dry” sample, sometimes referred to as a “core” diameter (that is, not in a solution where the surrounding solvent is interacting with the particles—see hydrodynamic diameter, above). As a non-limiting example, the diameter is determined using microscopic measurement techniques (for example, TEM or SEM). For a detailed discussion of the difference between core diameter and hydrodynamic diameter, see, e.g., Sci Technol Adv Mater. 2018; 19, 732-745.

When the amount of an impurity is specified at a level of “about” a value (e.g., “about 0,” “about 45%,” or “about 1300 nm”), it is understood by those of skill in the art that such a measurement is accurate to a certain number of significant figures based on the relevant detection method used.

### EXAMPLES

The invention now being generally described, it will be more readily understood by reference to the following examples which are included merely for purposes of illustration of certain aspects and embodiments of the present invention, and are not intended to limit the invention.

### Example 1: Exemplary Synthesis of Zno Compositions at 20 L Scale

A metal precursor solution and a buffer solution were prepared as follows:

### Buffer Solution—Vessel A:

In a 20-L vessel the following solutions were added:
900 mL of 1M solution of TRIS (pH~11);
18 L of reverse osmosis deionized (RODI) Water to diluted down the original solution to 50 mM;
   which was mixed vigorously with an industrial mixer to yield a colorless solution having a temperature of ~22-24 °C. Reactions may also be carried out with the buffer solution at temperatures from about 12 °C to about 32 °C, for example.

### Metal Precursor Solution—Vessel B:

1 mole of Zinc chloride (~136 g) was added to a 2-L filter flask containing 2-L of RODI water, yielding a colorless solution having a temperature of ~60 °C-65 °C. Reactions may also be carried out with the metal precursor solution at temperatures from about 45 °C to 90 °C, for example.

### Procedure

Content from Vessel B was slowly and continuously added to solution contained in Vessel A through the side arm of the filter flask. Upon mixing, the solution turned white immediately and the precipitate progressively started slowly precipitating. In about 1 hour, all the visible precipitate settled down at the bottom of the vessel. The pH of the solution where the product has formed—after the reaction takes place—drops to a value from about 7.5 to about 8.5. The white precipitated powder was collected by allowing it to pass through a trap at the bottom of the vessel and was collected in a secondary container where the powder and residual supernatant gets filtered using a large Buchner funnel sitting on a 2-L filter flask. Vacuum is applied by an electric vacuum pump.

The amount of water removed by vacuum filtration is proportional to the desired slurry type. For example, 10 wt %, 20 wt %, 30 wt %, 40 wt %, 50 wt %, 60 wt %, 70 wt %, and 80% water/ZnO slurries can be prepared. Percent water of aforementioned slurries were calculated by measuring the weight of approximately 1.5 g of the wet mixture before and after placing it in a desiccator overnight (10 hours at 156 F).

The white powder was characterized by TEM (accurate size), SEM (3D-topology), EDAX (elemental analysis), UV-Vis spectroscopy (absorbance shift), and DLS (size distribution).

The protocol above, with minor and routine adjustments, can be used to yield the following oxides:
Calcium Oxide (1M calcium nitrate tetrahydrate in Vessel B)
Chromium Oxide (1M chromium (II) chloride or chromium chloride tetrahydrate in Vessel B)
Europium Oxide (1M europium nitrate hexahydrate in Vessel B)
Iron Oxide [1M iron (II) chloride in Vessel B]
Lithium Oxide (1M Lithium iodide or lithium chloride in Vessel B)
Magnesium Oxide (1M magnesium nitrate or magnesium chloride hexahydrate in Vessel B)
Manganese Oxide [1M manganese (II) chloride in Vessel B]
Silver Oxide (1M silver nitrate in Vessel B)
Vanadium Oxide [1M vanadium (II) chloride in Vessel B]

In general, after mixing the metal precursor solution with the buffer solution, all oxide products that are insoluble in water will precipitate from resulting solution.

Any of these oxides can be co-synthesized with any other of the oxides, e.g. using the doping procedure set forth below in Example 2.

### Example 2: Synthesis of Exemplary Doped Zno Compositions

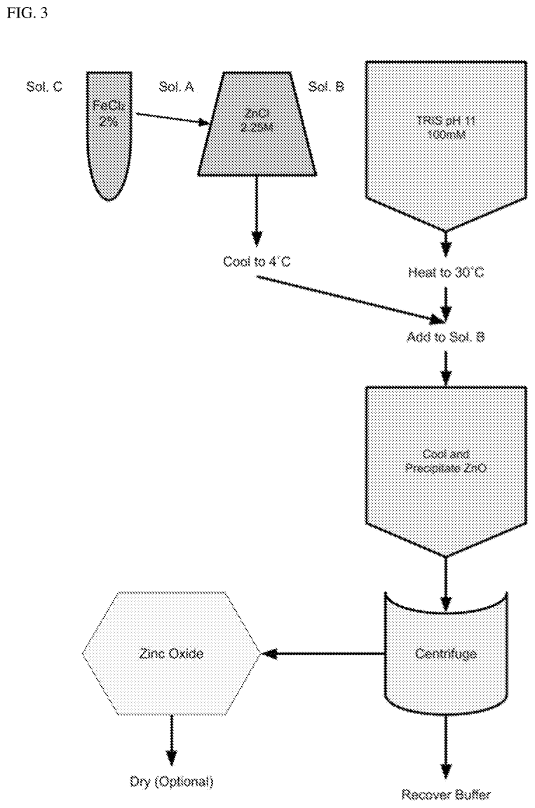

The protocol set forth in Example 1 can be readily modified to allow for any metal doped ZnO composition at any desired wt % dopant by adding one or more dopant precursors to the solution in Vessel B and following the procedure outlined in Example 1, above. (see also, FIG. 3). The following exemplary metal precursor solutions were prepared for use in the synthetic procedure of Example 1 to obtain doped ZnO compositions.

### Iron-Doped ZnO

1 mole of Zinc chloride (~136 g) and 0.05 moles of FeCl₂ (for a 5% Fe-doped product) were added to a 2-L filter flask containing 2-L of RODI water, yielding a colorless solution having a temperature of ~60 °C-65 °C. Reactions may also be carried out with the metal precursor solution at temperatures from about 45 °C to 90 °C, for example.

### Silver-Doped ZnO

1 mole of Zinc chloride (~136 g) and 0.05 moles of Ag(NO₃) (for a 5% Ag-doped product) were added to a 2-L filter flask containing 2-L of RODI water, yielding a colorless solution having a temperature of ~60 °C-65 °C. Reactions may also be carried out with the metal precursor solution at temperatures from about 45 °C to 90 °C, for example.

### Silver-and-Iron-Doped ZnO

1 mole of Zinc chloride (~136 g), 0.05 moles of Fe(NO₃)₂·6(H₂O) and 0.05 moles of Ag(NO₃) (for a 5% Ag/5% Fe-doped product) were added to a 2-L filter flask containing 2-L of RODI water, yielding a colorless solution having a temperature of ~60 °C-65 °C. Reactions may also be carried out with the metal precursor solution at temperatures from about 45 °C to 90 °C, for example.

### Example 3: Dynamic Light Scattering Data Collection Methods

Dry powder of zinc oxide composition was crushed in a mortar with a pestle only used for zinc oxide sample grinding. DLS samples were prepared by adding 1 mg of grinded zinc oxide in 1 mL of water. The suspension was sonicated for 10 seconds and diluted down by a factor of 5. 1.5 mL of the sample were placed in a cuvette and inserted in a Malvern Zetasizer (Nano ZS). It is fair to assume for the purpose of calculations that the water viscosity was not affected by the diluted suspension. The graph correlation showed optimal dispersion and narrow distribution of sizes was observed.

### Example 4: Transmission Electron Microscopy Data Collection Methods

All images were taken using a JEOL 2100 transmission electron microscope (TEM, 200 kV) equipped with a Gatan Digital camera and an EDAX-EDS (energy dispersive X-ray analysis/energy dispersive X-ray spectroscopy) system. The images were taken at the eucentric point to ensure reproducibility of the measurements. EDAX-EDS was performed on the same samples to identify elemental composition of the specimens.

Dry powder of zinc oxide was crushed in a mortar with a pestle only used for zinc oxide sample grinding. The powder was then transferred onto a clean glass slide and dispersed onto the cleaned, curved portion of a pipette bulb by gently rolling the bulb across the zinc oxide powder. The bulb was then rolled, in one pass, across the carbon-coated copper TEM grid (Ted Pella).

The “dry” delivery of the sample to the TEM grid was chosen to rule out any possible artifact due to traces of un-associated solvent molecules.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A dispersible powder composition, comprising:
metal oxide particles; and
water molecules;
wherein:
a portion of the water molecules are associated with the surface of the metal oxide particles.

2. The dispersible powder composition of claim 1, comprising:
metal oxide particles; and
water molecules;
wherein:
the water molecules comprise about 10 wt % to about 80 wt % of the composition; and
a portion of the water molecules are associated with the surface of the metal oxide particles.

3. The dispersible powder composition of claim 1, comprising:
metal oxide particles; and
water molecules;
wherein:
a portion of the water molecules are associated with the surface of the metal oxide particles; and
the metal oxide particles have a hydrodynamic diameter from about 15 nm to about 1500 nm.

4. The dispersible powder composition of claim 1, comprising:
metal oxide particles; and
water molecules;
wherein:
a portion of the water molecules are associated with the surface of the metal oxide particles; and
the metal oxide particles have a diameter from about 15 nm to about 1300 nm.

5. The dispersible powder composition of claim 1, comprising:
metal oxide particles; and
water molecules;
wherein:
a portion of the water molecules are associated with the surface of the metal oxide particles; and
the metal oxide particles have a diameter distribution characterized by a D90 of less than about 1200 nm.

6. The dispersible powder composition of claim 1, comprising:
metal oxide particles; and
water molecules;
wherein:
a portion of the water molecules are associated with the surface of the metal oxide particles; and
the metal oxide particles have a hydrodynamic diameter distribution characterized by a D90 of less than about 1300 nm.

7. The dispersible powder composition of any one of claims 1-6, wherein the metal oxide particles comprise a metal selected from a Group 1 metal, a Group 2 metal, a Group 3 metal, a Group 5 metal, a Group 6 metal, a Group 7 metal, a Group 8 metal, a Group 11 metal, and a Group 12 metal.

8. The dispersible powder composition of any one of claims 1-7, wherein the metal oxide particles comprise Li, Mg, Ca, Eu, V, Cr, Mn, Fe, Zn, Ag, or a combination thereof.

9. The dispersible powder composition of any one of claims 1-8, wherein the metal oxide particles comprise Fe.

10. The dispersible powder composition of any one of claims 1-9, wherein the metal oxide particles comprise Zn.

11. The dispersible powder composition of any one of claims 1-10, wherein the metal oxide particles do not comprise Ba.

12. The dispersible powder composition of any one of claims 1-11, wherein the metal oxide particles comprise at least two metallic elements.

13. The dispersible powder composition of claim 12, wherein the at least two metallic elements are each independently selected from a Group 1 metal, a Group 2 metal, a Group 3 metal, a Group 5 metal, a Group 6 metal, a Group 7 metal, a Group 8 metal, a Group 11 metal, and a Group 12 metal.

14. The dispersible powder composition of claim 12 or 13, wherein the at least two metallic elements are each independently selected from Li, Mg, Ca, Eu, V, Cr, Mn, Fe, Zn, and Ag.

15. The dispersible powder composition of any one of claims 1-14, wherein the metal oxide particles further comprise at least one dopant, and the dopant is a monoatomic cation.

16. The dispersible powder composition of claim 15, wherein the at least one dopant is a metallic element.

17. The dispersible powder composition of claim 15, wherein the at least one dopant is a semi-metallic element.

18. The dispersible powder composition of claim 15, wherein the at least one dopant is a nonmetallic element.

19. The dispersible powder composition of claim 15 or 16, wherein the at least one dopant is iron.

20. The dispersible powder composition of claim 15 or 16, wherein the at least one dopant is silver.

21. The dispersible powder composition of claim 15 or 16, wherein the at least one dopant is lithium.

22. The dispersible powder composition of claim 15 or 16, wherein the at least one dopant is a combination of iron and silver.

23. The dispersible powder composition of any one of claims 15-22, wherein the at least one dopant is present in an amount of from about 0.01 wt % to about 50 wt % relative to a total weight of the dispersible powder composition.

24. The dispersible powder composition of any one of claims 15-23, wherein the at least one dopant is present in an amount of about 0.5 wt %, about 1 wt %, about 2 wt %, or about 10 wt %.

25. The dispersible powder composition of any one of claims 1-24, wherein the dispersible powder composition does not comprise carbon.

26. The dispersible powder composition of any one of claims 1-25, wherein the dispersible powder composition does not comprise silicon.

27. The dispersible powder composition of any one of claims 1-26, wherein the metal oxide particles do not comprise a coating.

28. The dispersible powder composition of claim 1-27, wherein the metal oxide particles do not comprise a hydrophobic coating.

29. The dispersible powder composition of claim 1-27, wherein the metal oxide particles do not comprise a hydrophilic coating.

30. The dispersible powder composition of any one of claims 1-29, wherein the metal oxide particles do not comprise a coating comprising an organic dispersant.

31. The dispersible powder composition of any one of claims 1-30, wherein only the surface-associated water molecules are associated with the surface of the metal oxide particles.

32. The dispersible powder composition of any one of claims 1-31, wherein the surface of the metal oxide particles has not been functionalized or modified by the addition of functional groups.

33. The dispersible powder composition of claim 32, wherein the functional groups comprise alkyl, alkenyl, alkynyl, alkoxy, siloxy, silyl, carboxyl, amino, or phosphoalkyl, or a combination thereof.

34. The dispersible powder composition of any one of claims 1-33, wherein the metal oxide particles are in the form of a non-spherical aggregate having an increased surface area relative to a sphere of the same diameter.

35. The dispersible powder composition of any one of claims 1-34, wherein the metal oxide particles are in the form of a rounded, cauliflower-like shape.

36. The dispersible powder composition of any one of claims 1-35, wherein the metal oxide particles have a form substantially as shown in FIG. 5.

37. The dispersible powder composition of any one of claims 1-36, wherein the dispersible powder composition has a maximum optical absorption wavelength (λₘₐₓ) at about 375 nm.

38. The dispersible powder composition of any one of claims 1-37, wherein the dispersible powder composition has a maximum optical absorption wavelength (λₘₐₓ) at about 375 nm, with a molar absorption coefficient (ϵ) of about 5×10⁻⁴ M⁻¹ ·cm⁻¹.

39. The dispersible powder composition of any one of claims 1 and 3-38, wherein the total number of water molecules comprise about 10 wt % to about 80 wt % of the composition.

40. The dispersible powder composition of any one of claims 1-39, wherein the total number of water molecules comprise about 30 wt % to about 70 wt % of the composition.

41. The dispersible powder composition of any one of claims 1-40, wherein the total number of water molecules comprise about 30 wt % to about 40 wt % of the composition.

42. The dispersible powder composition of any one of claims 1-40, wherein the total number of water molecules comprise about 41 wt % to about 50 wt % of the composition.

43. The dispersible powder composition of any one of claims 1-40, wherein the total number of water molecules comprise about 51 wt % to about 60 wt % of the composition.

44. The dispersible powder composition of any one of claims 1-40, wherein the total number of water molecules comprise about 61 wt % to about 70 wt % of the composition.

45. The dispersible powder composition of any one of claims 1, 2, and 4-44, wherein the metal oxide particles have a hydrodynamic diameter from about 15 nm to about 1500 nm.

46. The dispersible powder composition of any one of claims 1-45, wherein the metal oxide particles have a hydrodynamic diameter from about 100 nm to about 1500 nm.

47. The dispersible powder composition of claim 45 or 46, wherein the metal oxide particles have a hydrodynamic diameter of from about 300 to about 1150 nm.

48. The dispersible powder composition of any one of claims 3 and 45-47, wherein the hydrodynamic diameter is measured by Dynamic Light Scattering or Photon Correlation Spectroscopy.

49. The dispersible powder composition of claim 48, the hydrodynamic diameter is measured by Dynamic Light Scattering.

50. The dispersible powder composition of any one of claims 1-3 and 5-36, wherein the metal oxide particles have a diameter from about 15 nm to about 1300 nm.

51. The dispersible powder composition of any one of claims 1-50, wherein the metal oxide particles have a diameter from about 100 nm to about 1250 nm.

52. The dispersible powder composition of any one of claims 1-51, wherein the metal oxide particles have a diameter from about 200 nm to about 920 nm.

53. The dispersible powder composition of any one of claims 4 and 50-52, wherein the diameter is measured by Transmission Electron Microscopy (TEM).

54. The dispersible powder composition of any one of claims 4 and 50-52, wherein the diameter is measured by Scanning Electron Microscopy (SEM).

55. The dispersible powder composition of any one of claims 1-4 and 6-54, wherein the metal oxide particles have a diameter distribution characterized by a D90 of less than about 1200 nm.

56. The dispersible powder composition of claim 55, wherein the diameter distribution is characterized by a D90 from about 5 nm to about 1200 nm.

57. The dispersible powder composition of any one of claims 1-56, wherein the metal oxide particles have a diameter distribution characterized by a D50 of less than about 650 nm.

58. The dispersible powder composition of claim 57, wherein the diameter distribution is characterized by a D50 from about 5 nm to about 650 nm.

59. The dispersible powder composition of any one of claims 1-58, wherein the metal oxide particles have a diameter distribution characterized by a D10 of less than about 25 nm.

60. The dispersible powder composition of claim 59, wherein the diameter distribution is characterized by a D10 from about 5 nm to about 25 nm.

61. The dispersible powder composition of any one of claims 1-5 and 7-60, wherein the metal oxide particles have a hydrodynamic diameter distribution characterized by a D90 of less than about 1300 nm.

62. The dispersible powder composition of claim 61, wherein the hydrodynamic diameter distribution is characterized by a D90 from about 5 nm to about 1300 nm.

63. The dispersible powder composition of any one of claims 1-62, wherein the metal oxide particles have a hydrodynamic diameter distribution characterized by a D50 of less than about 700 nm.

64. The dispersible powder composition of claim 63, wherein the hydrodynamic diameter distribution is characterized by a D50 from about 5 nm to about 700 nm.

65. The dispersible powder composition of any one of claims 1-64, wherein the metal oxide particles have a hydrodynamic diameter distribution characterized by a D10 of less than about 150 nm.

66. The dispersible powder composition of any one of claims 1-65, wherein the hydrodynamic diameter distribution is characterized by a D10 from about 5 nm to about 150 nm.

67. An aqueous composition comprising the dispersible powder composition of any one of claims 1-66 and an aqueous carrier.

68. The aqueous composition of claim 67, wherein the aqueous carrier is a pharmaceutically acceptable carrier or a cosmetically acceptable carrier.

69. The aqueous composition of claim 67 or 68, wherein the aqueous carrier is selected from water, biocompatible buffers, saline solutions, a gel base (e.g., a hydrogel), and micellar water.

70. A method of preparing the composition of any one of claims 1-66 or the aqueous composition of any one of claims 67-69 the method comprising:
dissolving at least one metal precursor in a first aqueous solution to afford a metal precursor solution;
adding the metal precursor solution to a buffer solution comprising water and a buffer, thereby affording a combined solution;
stirring the combined solution until the dispersible powder composition precipitates from the combined solution; and
optionally separating the dispersible powder composition from the combined solution;
wherein the difference in temperature between the buffer solution and the metal precursor solution is from about 13 °C to about 78 °C.

71. A method for preparing a dispersible powder composition, the composition comprising:
metal oxide particles; and
water molecules;
wherein:
a portion of the water molecules are associated with the surface of the metal oxide particles;
the method comprising:
dissolving at least one metal precursor in a first aqueous solution to afford a metal precursor solution;
adding the metal precursor solution to a buffer solution comprising water and a buffer, thereby affording a combined solution;
mixing the combined solution thereby causing the dispersible powder composition to precipitate from the combined solution; and
optionally separating the dispersible powder composition from the combined solution;
wherein the difference in temperature between the buffer solution and the metal precursor solution is from about 13 °C to about 78 °C.

72. The method of claim 71, wherein the first aqueous solution comprises reverse osmosis deionized water.

73. The method of claim 71 or 72, wherein the method comprises separating the dispersible powder composition from the combined solution.

74. The method of any one of claims 71-73, wherein the difference in temperature between the buffer solution and the metal precursor solution is from about 15 °C to about 50 °C.

75. The method of any one of claims 71-74, wherein the difference in temperature between the buffer solution and the metal precursor is from about 20 °C to about 30 °C.

76. The method of any one of claims 71-75, wherein the difference in temperature between the buffer solution and the metal precursor is about 25 °C.

77. The method of any one of claims 71-76, wherein the temperature of the metal precursor solution is from about 40 °C to about 90 °C when added to the buffer solution.

78. The method of any one of claims 71-77, wherein the temperature of the metal precursor solution is about 60 °C to about 65 °C when added to the buffer solution.

79. The method of any one of claims 71-78, wherein the buffer solution is at a buffer solution temperature from about 12 °C to about 32 °C when the metal precursor solution is added.

80. The method of any one of claims 71-79, wherein the buffer solution is at a buffer solution temperature of about 22 °C to about 24 °C when the metal precursor solution is added.

81. The method of any one of claims 71-80, wherein each of the at least one metal precursors is a neutral ionic compound comprising a metal cation and at least one counter-anion.

82. The method of claim 81, wherein the metal cation comprises a metal selected from a Group 1 metal, a Group 2 metal, a Group 3 metal, a Group 5 metal, a Group 6 metal, a Group 7 metal, a Group 8 metal, a Group 11 metal, and a Group 12 metal.

83. The method of claim 81 or 82, wherein the metal cation comprises Li, Mg, Ca, Eu, V, Cr, Mn, Fe, Zn or Ag, or a combination thereof.

84. The method of any one of claims 81-83, wherein the metal cation comprises Zn.

85. The method of any one of claims 81-84, wherein the metal cation is Zn²⁺.

86. The method of any one of claims 81-85, wherein the metal cation does not comprise Ba.

87. The method of any one of claims 71-86, wherein the at least one metal precursor is

88. The method of any one of claims 71-86, wherein the at least one metal precursor is at least two metal precursors, and each of the at least two metal precursors is a neutral ionic compound comprising a metal cation and at least one counter-anion.

89. The method of claim 88, wherein the at least two metal precursors each independently comprise a metal cation of a Group 1 metal, a Group 2 metal, a Group 3 metal, a Group 5 metal, a Group 6 metal, a Group 7 metal, a Group 8 metal, a Group 11 metal, a Group 12 metal, or a combination thereof.

90. The method of claim 88 or 89, wherein the metal cation is selected from Li, Mg, Ca, Eu, V, Cr, Mn, Fe, and Ag.

91. The method of any one of claims 71-90, further comprising dissolving at least one dopant precursor in the first aqueous solution, wherein the at least one dopant precursor is a neutral ionic compound comprising at least one dopant and at least one counter-anion, and wherein the dopant is a monoatomic cation.

92. The method of claim 91, wherein the at least one dopant is a metallic element.

93. The method of claim 91, wherein the at least one dopant is a semi-metallic element.

94. The method of claim 91, wherein the at least one dopant is a nonmetallic element.

95. The method of claim 91 or 92, wherein the at least one dopant is iron.

96. The method of claim 91 or 92, wherein the at least one dopant is silver.

97. The method of claim 91 or 92, wherein the at least one dopant is lithium.

98. The method of claim 91 or 92, wherein the at least one dopant is iron and silver.

99. The method of any one of claims 91-98, wherein the at least one dopant is present in an amount of from about 0.01 wt % to about 50 wt % relative to a total weight of the dispersible powder composition.

100. The method of any one of claims 81-99, wherein the at least one counter-anion is selected from halide, nitrate, and acetate.

101. The method of any one of claims 81-100, wherein the at least one counter-anion is nitrate.

102. The method of any one of claims 81-100, wherein the at least one counter-anion is chloride, bromide, or iodide.

103. The method of any one of claims 81-100 and 102, wherein the at least one counter-anion is chloride.

104. The method of any one of claims 81-103, wherein the neutral ionic compound is selected from: ZnCl₂, ZnBr₂, Zn(NO₃)₂, FeCl₂, FeCl₃, Fe(NO₃)₂, Fe(NO₃)₃, AgCl, Ag(NO₃), CrCl₂, Eu(NO₃)₃, Lil, LiCl, Li(NO₃), MgCl₂, MgBr₂, Mg(NO₃)₂, MnCl₂, MnBr₂, Mn(NO₃)₂, VCl₂, VBr₂, and V(NO₃)₂, or a hydrate thereof.

105. The method of any one of claims 71-104, wherein the at least one metal precursor is water-soluble.

106. The method of claim 105, wherein the metal precursor has a solubility of at least about 0.01 mol/L in water at 25 °C.

107. The method of any one of claims 71-106, wherein the at least one metal precursor is a hydrate.

108. The method of any one of claims 71-107, wherein each of the at least one metal precursors solution has a concentration from about 0.1 M to about 5 M.

109. The method of any one of claims 71-108, wherein the concentration of the metal precursor in the metal precursor solution is from about 0.5 M to about 1.5 M.

110. The method of any one of claims 71-109, wherein the concentration of the metal precursor in the metal precursor solution is about 0.5 M, about 0.75 M, about 1.0 M, about 1.25 M, or about 1.5 M.

111. The method of any one of claims 71-110, wherein the buffer solution has a pH greater than about 7.5.

112. The method of any one of claims 71-111, wherein the buffer solution has a pH of about 8.5.

113. The method of any one of claims 71-111, wherein the buffer solution has a pH greater than about 9.

114. The method of any one of claims 71-111 and 113, wherein the buffer solution has a pH of about 10.

115. The method of any one of claims 71-111 and 113, wherein the buffer solution has a pH of about 11.

116. The method of any one of claims 71-115, wherein the water in the buffer solution comprises reverse osmosis deionized water.

117. The method of any one of claims 71-116, wherein the buffer is biocompatible.

118. The method of any one of claims 71-117, wherein the buffer is non-toxic.

119. The method of any one of claims 71-118, wherein the buffer is selected from: tris(hydroxymethyl)aminomethane (TRIS), borate (BO₃³⁻), TE (TRIS and EDTA), phosphates (e.g., PBS), and salts thereof.

120. The method of any one of claims 117-119, wherein the concentration of the buffer in the buffer solution is about 10 mM to 100 mM.

121. The method of any one of claims 117-120, wherein the concentration of the buffer in the buffer solution is about 10 mM, about 20 mM, about 30 mM, about 40 mM, about 50 mM, about 60 mM, about 70 mM, about 80 mM, about 90 mM, or about 100 mM.

122. The method of any one of claims 71-121, wherein the combined solution has a pH from about 7.5 to about 8.5 when the dispersible powder composition precipitates from the combined solution.

123. The method of any one of claims 71-122, wherein the dispersible powder composition comprises from about 10 wt. % to about 60 wt. % water.

124. The method of any one of claims 71-123, wherein the dispersible powder composition comprises about 10 wt. %, about 20 wt. %, about 30 wt. %, about 40 wt. %, about 50 wt. %, or about 60 wt. % water.

125. The method of any one of claims 71-124, further comprising drying the dispersible powder composition, thereby removing a portion of the water molecules not associated with the surface of the metal oxide particles.

126. The method of claim 125, wherein drying the dispersible powder composition involves desiccating the dispersible powder composition.

127. The method of any one of claims 71-125, wherein the at least one dopant is present in an amount of about 0.5 wt %, about 1 wt %, about 2 wt %, or about 10 wt %.

128. The method of any one of claims 71-127, wherein the dispersible powder composition does not comprise carbon.

129. The method of any one of claims 71-128, wherein the dispersible powder composition does not comprise silicon.

130. The method of any one of claims 71-129, wherein the metal oxide particles do not comprise a coating.

131. The method of claim 130, wherein the metal oxide particles do not comprise a hydrophobic coating.

132. The method of claim 130, wherein the metal oxide particles do not comprise a hydrophilic coating.

133. The method of any one of claims 71-132, wherein the metal oxide particles do not comprise a coating comprising an organic dispersant.

134. The method of any one of claims 71-133, wherein only the surface-associated water molecules are associated with the surface of the metal oxide particles.

135. The method of any one of claims 71-134, wherein the surface of the metal oxide particles has not been functionalized or modified by the addition of functional groups.

136. The method of any one of claims 71-135, wherein the functional groups comprise alkyl, alkenyl, alkynyl, alkoxy, siloxy, silyl, carboxyl, amino, or phosphoalkyl, or a combination thereof.

137. The method of any one of claims 71-136, wherein the metal oxide particles are in the form of a non-spherical aggregate having an increased surface area relative to a sphere of the same diameter.

138. The method of any one of claims 71-137, wherein the metal oxide particles are in the form of a rounded, cauliflower-like shape.

139. The method of any one of claims 71-138, wherein the metal oxide particles have a form substantially as shown in FIG. 5.

140. The method of any one of claims 71-139, wherein the dispersible powder composition has a maximum optical absorption wavelength (λₘₐₓ) at about 375 nm.

141. The method of any one of claims 71-140, wherein the dispersible powder composition has a maximum optical absorption wavelength (μₘₐₓ) at about 375 nm, with a molar absorption coefficient (ϵ) of about 5×10⁻⁴ M⁻¹·cm⁻¹.

142. The method of any one of claims 71-141, wherein the total number of water molecules comprise about 10 wt % to about 45 wt % of the composition.

143. The method of any one of claims 71-142, wherein the total number of water molecules comprise about 30 wt % to about 70 wt % of the composition.

144. The method of any one of claims 71-143, wherein the total number of water molecules comprise about about 30 wt % to about 40 wt % of the composition.

145. The method of any one of claims 71-143, wherein the total number of water molecules comprise about 41 wt % to about 50 wt % of the composition.

146. The method of any one of claims 71-143, wherein the total number of water molecules comprise about 51 wt % to about 60 wt % of the composition.

147. The method of any one of claims 71-143, wherein the total number of water molecules comprise about 61 wt % to about 70 wt % of the composition.

148. The method of any one of claims 71-147, wherein the metal oxide particles have a hydrodynamic diameter from about 15 nm to about 1500 nm.

149. The method of any one of claims 71-148, wherein the metal oxide particles have a hydrodynamic diameter from about 100 nm to about 1500 nm.

150. The method of any one of claims 71-149, wherein the metal oxide particles have a hydrodynamic diameter of from about 300 to about 1150 nm.

151. The method of any one of claims 71-150, wherein the hydrodynamic diameter is measured by Dynamic Light Scattering or Photon Correlation Spectroscopy.

152. The method of any one of claims 71-151, wherein the hydrodynamic diameter is measured by Dynamic Light Scattering.

153. The method of any one of claims 71-152, wherein the metal oxide particles have a diameter from about 15 nm to about 1300 nm.

154. The method of any one of claims 71-153, wherein the metal oxide particles have a diameter from about 100 nm to about 1250 nm.

155. The method of any one of claims 71-154, wherein the metal oxide particles have a diameter from about 200 nm to about 920 nm.

156. The method of any one of claims 71-155, wherein the diameter is measured by Transmission Electron Microscopy (TEM).

157. The method of any one of claims 71-155, wherein the diameter is measured by Scanning Electron Microscopy (SEM).

158. The method of any one of claims 71-157, wherein the metal oxide particles have a diameter distribution characterized by a D90 of less than about 1200 nm.

159. The method of claim 158, wherein the diameter distribution is characterized by a D90 from about 5 nm to about 1200 nm.

160. The method of any one of claims 71-159, wherein the metal oxide particles have a diameter distribution characterized by a D50 of less than about 650 nm.

161. The method of claim 160, wherein the diameter distribution is characterized by a D50 from about 5 nm to about 650 nm.

162. The method of any one of claims 71-161, wherein the metal oxide particles have a diameter distribution characterized by a D10 of less than about 25 nm.

163. The method of claim 162, wherein the diameter distribution is characterized by a D10 from about 5 nm to about 25 nm.

164. The method of any one of claims 71-163, wherein the metal oxide particles have a hydrodynamic diameter distribution characterized by a D90 of less than about 1300 nm.

165. The method of claim 164, wherein the hydrodynamic diameter distribution is characterized by a D90 from about 5 nm to about 1300 nm.

166. The method of any one of claims 71-165, wherein the metal oxide particles have a hydrodynamic diameter distribution characterized by a D50 of less than about 700 nm.

167. The method of claim 166, wherein the hydrodynamic diameter distribution is characterized by a D50 from about 5 nm to about 700 nm.

168. The method of any one of claims 71-167, wherein the metal oxide particles have a hydrodynamic diameter distribution characterized by a D10 of less than about 150 nm.

169. The method of claim 168, wherein the hydrodynamic diameter distribution is characterized by a D10 from about 5 nm to about 150 nm.

170. A dispersible powder composition prepared by the method of any one of claims 71-169.
